(19) United States
(12) Patent Application Publication
(10) Pub. No.: US 2017/0181660 A1
(43) Pub. Date: Jun. 29, 2017

(54) METHODS TO OBTAIN CROSS-SECTIONAL AREAS WITHIN LUMINAL ORGANS USING IMPEDANCE

(71) Applicant: 3DT Holdings, LLC, San Diego, CA (US)

(72) Inventor: Ghassan S. Kassab, La Jolla, CA (US)

(73) Assignee: 3DT Holdings, LLC, San Diego, CA (US)

(21) Appl. No.: 15/400,737

(22) Filed: Jan. 6, 2017

Related U.S. Application Data
(63) Continuation of application No. 13/520,944, filed on Jul. 6, 2012.
(60) Provisional application No. 61/293,086, filed on Jan. 7, 2010.

(51) Int. Cl. A61B 5/053; A61B 5/107

(52) U.S. Cl. CPC A61B 5/0538; A61B 5/1076

(57) ABSTRACT

Systems and methods to obtain parallel tissue conductances within luminal organs. In one method embodiment to obtain a parallel tissue conductance within a luminal organ of the present disclosure, the method comprises the steps of introducing at least part of a detection device into a luminal organ at a first location, the detection device having a detector, applying current to the detection device using a stimulator, and introducing a first signal having a first frequency and a second signal having a second frequency through the detection device.

### PRIORITY

The present application is related to, claims the priority benefit of, and is a U.S. continuation application of, U.S. patent application Ser. No. 13/520.944. filed Jul. 6, 2012, which is related to. claims the priority benefit of, and is a U.S. § 371 national state application of, international patent application Ser. No. PCT/US11/20532, filed Jan. 7, 2011, which is related to, and claims the priority benefit of, U.S. Provisional Patent Application Ser. No. 61/293,086, filed Jan. 7, 2010. The entire contents of each of the foregoing applications are hereby incorporated by reference into this disclosure.

### BACKGROUND

Coronary heart disease (CHD) is commonly caused by atherosclerotic narrowing of the coronary arteries and is likely to produce angina pectoris, heart attacks or a combination. CHD caused 466,101 deaths in the USA in 1997 and is one of the leading causes of death in America today. To address CHD, intra-coronary stents have been used in large percentages of CHD patients. Stents increase the minimal coronary lumen diameter to a greater degree than percutaneous transluminal coronary angioplasty (PTC A) alone.

Intravascular ultrasound is a method of choice to determine the true diameter of a diseased vessel in order to size the stent correctly. The tomographic orientation of ultrasound enables visualization of the fall 360° circumference of the vessel wall and permits direct measurements of lumen dimensions, including minimal, and maximal diameter and cross-sectional area. Information front ultrasound is combined with that obtained by angiography. Because of the latticed characteristics of stents, radiographic contrast material can surround the stent, producing an angiographic appearance of a large lumen, even when the stent struts are not in full contact with the vessel wall. A large observational ultrasound study after angio-graphically guided, stent deployment revealed an average residual plaque area of 51% in a comparison of minimal stent diameter with reference segment diameter, and incomplete wall apposition was frequently observed. In this cohort, additional balloon inflations resulted in a final average residual plaque area of 34%, even though the final angiographic percent stenosis was negative (20.7%). Those investigators used ultrasound to guide deployment.

However, using intravascular ultrasound as mentioned above requires a first step of advancement of an ultrasound catheter and then withdrawal of the ultrasound catheter before coronary angioplasty thereby adding additional time to the stent procedure. Furthermore, it requires an ultrasound machine. This adds significant cost and time and more risk to the procedure.

One common type of coronary artery disease is atherosclerosis, which is a systemic inflammatory disease of the vessel wall that affects multiple arterial beds, such, as aorta, carotid and peripheral arteries, and causes multiple coronary artery lesions and plaques. Atheroselerotic plaques typically include connective tissue, extracellular matrix (including collagen, proteoglycans, and fibronectin elastic fibers), lipid (crystalline cholesterol, cholesterol esters and phospholipids), and cells such as monocyte-derived macrophages, T lymphocytes, and smooth muscles cells. A wide range of plaques occurs pathologically with varying composition of these components.

A process called “positive remodeling” occurs early on during the development of atherosclerosis in coronary artery disease (CAD) where the lumen cross-sectional area (CSA) stays relatively normal because of the expansion of external elastic membrane and the enlargement of the outer CSA. However, as CAD progresses, there is no further increase in the external diameter of the external elastic membrane. Instead, the plaque begins to impinge into the lumen and decreases the lumen CSA in a process called “negative remodeling”.

Evidence shows that that a non-significant coronary atherosclerotic plaque (typically <50% stenosis) can rupture and produce myocardial infarct even before it produces significant lumen narrowing if the plaque has a particular composition. For example, a plaque with a high concentration of lipid and a thin fibrous cap may be easily sheared of ruptured and is referred to as a “vulnerable” plaque. In contrast, “white” plaques are less likely to rupture because the increased fibrous content over the lipid core provides stability (“stable” plaque). A large lipid core (typically >40%) rich in cholesterol is at a high risk for rupture and is considered a “vulnerable” plaque. In summary, plaque composition appears to determine the risk of acute coronary syndrome more so than the standard degree of stenosis because a higher lipid core is a basic characteristic, of a higher risk plaque.

Conventionally, angiography has been used to visualize and characterize atherosclerotic plaque in coronary arteries. Because of the recent finding that plaque composition, rather than severity of stenosis, determines the risk for acute coronary syndromes, newer imaging modalities are required to distinguish between and determine the composition of “stable” and “vulnerable” plaques. Although a number of invasive and noninvasive imaging techniques are available to assess atherosclerotic vessels, most of the standard techniques identify luminal diameter, stenosis, wall thickness and plaque volume. To date, there is no standard method that can characterize plaque composition (e.g., lipid, fibrous, calcium, or thrombus) and therefore there is no routine and reliable method to identify the higher risk plaques.

Noninvasive techniques for evaluation of plaque composition include magnetic resonance imaging (MRI). However, MRI lacks the sufficient spatial resolution, for characterization of the atherosclerotic lesion in the coronary vessel. Minimally invasive techniques for evaluation of plaque composition include intravascular ultrasound (IVUS), optical coherence tomography (OCT), raman and infrared spectroscopy. Thermography is also a catheter based technique used to detect the vulnerable plaques on the basis of temperature difference caused by the inflammation in the plaque. Using the various catheter-based techniques requires a first step of advancement of an IVUS, OCT, or thermography catheter and then withdrawal of the catheter before coronary angioplasty thereby adding additional time and steps to the stent procedure. Furthermore, these devices require expensive machinery and parts to operate. This adds significant cost and time and more risk to the procedure.

Thus, a need exists in the art for an alternative to the conventional methods of determining crass-sectional area of a luminal organ and determining the plaque-type of a plaque present within a luminal organ. A further need exist for a reliable, accurate and minimally invasive system or technique of determining the same.

### BRIEF SUMMARY

The disclosure of the present application provides various systems and methods for obtaining parallel tissue conductances within luminal organs. In at least one embodiment of a single solution injection method to obtain a parallel tissue conductance within a luminal organ of the present disclosure, the method comprises the steps of introducing at least part of a detection device into a luminal organ at a first location, the detection device having, a detector, applying current to the detection device using a stimulator, introducing a first signal having a first frequency and a second signal having a second frequency through the detection device, and injecting a solution having a known conductivity into the luminal organ at or near the detector of the detection device. Such a method may further comprise the steps of measuring an output conductance of the first signal and the second signal at the first location using the detector, and calculating, a parallel tissue conductance at the first location based in part upon the output conductance and the conductivity of the injected solution.

In at least another embodiment of a single solution injection method to obtain a parallel tissue conductance within a luminal organ of the present, disclosure, the method comprises the steps of introducing at least part of a detection device into a luminal organ at a first location, the detection device having a detector, applying current to the detection device using a stimulator, introducing a first signal having a first frequency and a second signal having a second frequency through the detection device, and measuring a first output conductance of the-first signal and the second signal at the first location in connection with a fluid native to the first location, said fluid having a first conductivity. An exemplary method may further comprise the steps of injecting a solution having a known conductivity into the luminal organ at or near the detector of the detection device, measuring a second output conductance of the first signal and the second signal at the first location in connection with the injected solution, and calculating a parallel tissue conductance at the first location based in part upon the second output conductance and the known conductivity of the injected solution.

In at least one embodiment of a single solution injection method to obtain a parallel tissue conductance within a luminal organ of the present disclosure, the step of calculating a parallel tissue conductance comprises the step of calculating a cross-sectional area of the luminal organ at the first location. In another embodiment, the step of introducing a first signal having a first frequency and a second signal having a second frequency is performed using a frequency generator. In an additional embodiment, the frequency generator comprises an arbitrary waveform generator. In yet an additional embodiment, the frequency generator comprises two signal generators.

In at least one embodiment of a single solution injection method to obtain a parallel tissue conductance within a luminal organ of the present disclosure, the output conductance comprises a first conductance value and a second conductance value. In an additional embodiment, the first conductance value corresponds to the first frequency and the second conductance value corresponds to the second frequency. In yet an additional embodiment, the step of calculating a cross-sectional area comprises the step of deconvoluting the output conductance to obtain a first conductance value and a second conductance value from the output conductance.

In at least one embodiment of a single solution injection method to obtain a parallel tissue conductance within a luminal organ of the present disclosure, the output conductance comprises a mixed signal. In another embodiment, the step of calculating a cross-sectional area further comprises the step of deconvoluting the mixed signal to obtain a first conductance value and a second conductance value from the mixed signal. In yet another embodiment, the first signal and the second signal are repeatedly alternated to form a multiplexed signal. In an additional embodiment, the first signal and the second signal are separated in time by less than 100 milliseconds. In yet an additional embodiment, the first signal and the second signal are separated in time by less than 10 milliseconds. In another embodiment, the first signal and the second signal are combined, to form a combined signal.

In at least one embodiment of a single solution injection method to obtain a parallel tissue conductance within a luminal organ of the present disclosure, the first location comprises a plaque site. In another embodiment, the step of calculating a parallel tissue conductance comprises the step of determining plaque-type composition of a plaque at the plaque site. In yet another embodiment, the luminal organ is selected from the group consisting of a body lumen, a body vessel, a blood vessel, a biliary tract, a urethra, and an esophagus. In an additional embodiment, the detector comprises two detection electrodes positioned in between two excitation electrodes, wherein the two excitation electrodes are capable of producing an electrical field. In yet another embodiment, the method further comprises the steps of moving the detection device to a second location within the luminal organ, injecting the solution into the luminal organ at or near the detector of the detection device, measuring a second output conductance of the first signal and the second signal at the second location using the detection device, calculating a second parallel tissue conductance at the second location based in part upon the output conductance and fee conductivity of the injected solution, calculating a second cross-sectional area of the luminal organ at the second location, and determining a profile of the luminal organ indicative of the first location and the second location based upon the calculated cross-sectional area and the calculated second cross-sectional area.

In at least one embodiment of a single solution injection, method to determine a cross-sectional area of a luminal organ of the present disclosure, the method comprises the steps of introducing at least part of a detection device into a luminal organ at a first location, the detection device having a detector, applying current to the detection device using a stimulator, introducing a first signal having a first frequency and a second signal having a second frequency through the detection device, injecting a solution having a known conductivity into the luminal organ at or near the detector of the detection device, measuring an output conductance of the first signal and the second signal at the first location using the detector, and calculating a cross-sectional area of the luminal organ at the first location based in part upon the output conductance and the conductivity of the injected solution.

In at least one embodiment of a single solution injection method to assess the composition of a plaque within a luminal organ of the present disclosure, the method comprises the steps of introducing at least part of a detection device into a luminal organ at a plaque site, the detection device having a detector, applying current to the detection device using a stimulator, introducing a first signal having a first frequency and a second signal having a second frequency through the detection device, injecting a solution having a known conductivity into the luminal organ at or near the detector of the detection device, measuring an output conductance of the first signal and the second signal at the plaque site using the detector, and determining plaque-type composition of a plaque at the plaque site based in part upon the output conductance and the conductivity of the injected solution.

In at least one embodiment of a single injection method to obtain a parallel tissue conductance within a luminal organ of the present disclosure, the method comprises the steps of introducing at least part of a detection device into a luminal organ at a first location, the detection device having a detector, applying current to the detection device using a stimulator, introducing a first signal having a first frequency and a second, signal having a second frequency through the detection device, measuring a first output conductance of the first signal and the second signal at the first location in connection with a fluid native to the first location using the detector, said fluid having a first conductivity, injecting a solution having a known conductivity into the luminal organ at or near the detector of the detection device, measuring a second output conductance of the first signal and the second signal at the first location in connection with the injected solution using the detector, and calculating a parallel tissue conductance al the first location based in part upon the second output conductance and the known conductivity of the injected solution. In another embodiment, the step of calculating the parallel tissue conductance is further based in part upon the first output conductance and file native conductivity of the native fluid. In yet another embodiment the step of calculating the parallel tissue conductance comprises the step of deconvoluting the second output conductance to obtain a first resulting conductance value and a second resulting conductance value from the second output conductance. In an additional embodiment, the step of calculating a parallel tissue conductance comprises the step of calculating a cross-sectional area of the luminal organ at the first location. In yet an additional embodiment, the first location comprises a plaque site. In another embodiment, the step of calculating a parallel tissue conductance comprises the step of determining plaque-type composition of a plaque at the plaque site.

In at least one embodiment of a single injection method to obtain a parallel tissue conductance within a luminal organ of the present disclosure, the method comprises the steps of introducing at least part of a detection device into a luminal organ at a first location, the detection, device having a detector, applying current to fee detection device, obtaining a first output conductance indicative of a bodily fluid native to the luminal organ, using the detector, injecting a solution having a known conductivity into the luminal organ at or near the detector of the detection device, measuring a second output conductance indicative of the injected solution using the detector, and calculating a parallel tissue conductance based in part upon the first output conductance, the second output conductance, and the known conductivity of the injected solution. In another embodiment, the step of calculating the parallel tissue conductance is farther based in part upon a conductivity of the bodily fluid native to the luminal organ. In yet another embodiment, the step of calculating the parallel tissue conductance farther comprises the step of calculating a cross-sectional area of the luminal organ at the first location. In an additional embodiment, the step of calculating the cross-sectional area is based in part upon a known distance between detection electrodes of the detector.

In at least one embodiment of a single injection method to obtain a parallel tissue conductance within a luminal organ of the present disclosure, the first output conductance is further indicative of a known diameter of a lumen defined within the detection device. In an additional embodiment, the first output conductance is further indicative of a known cross-sectional area of a lumen defined within, the detection device. In yet an additional embodiment, the first location comprises a plaque site. In another embodiment, the step of calculating the parallel tissue conductance further comprises the step of determining plaque-type composition of a plaque at the plaque site.

In at least one embodiment of a single injection method to obtain a parallel tissue conductance within a luminal organ of the present disclosure, the method further comprises the steps of moving the detection device to a second location within the luminal organ, injecting the solution into the luminal organ at or near the detector of the detection device, measuring a third output conductance indicative of the injected solution using the detector, calculating a second parallel tissue conductance based in part upon the first output conductance, the third output conductance, and the known conductivity of the injected solution, calculating a second cross-sectional area of the luminal organ at the second location, and determining a profile of the luminal organ indicative of the first location and the second location based upon the calculated cross-sectional area and the calculated second cross-sectional area.

In at least one embodiment of a single injection method to determine a cross-sectional area of a luminal organ of the present disclosure, the method comprises the steps of introducing at least part of a detection device into a luminal organ at a first location, the detection device having a detector, applying current to the detection device, obtaining a first output conductance indicative of a bodily fluid native to the luminal organ using the detector, injecting a solution having a known conductivity into the luminal organ at or near the detector of the detection device, measuring a second output conductance indicative of the injected solution using the detector, and calculating a cross-sectional area of the luminal organ at the first location based in part upon the first output conductance, the second output conductance, and the known conductivity of the injected solution, in another embodiment, the step of calculating the cross-sectional area is further based in part upon a conductivity of the bodily fluid native to the luminal organ. In yet another embodiment, the step of calculating the cross-sectional area is further based in part upon, a known distance between detection electrodes of the detector. In an additional embodiment, the first output conductance is further indicative of a known diameter of a lumen defined within the detection device. In yet an additional embodiment, the first output conductance is further indicative of a known cross-sectional area of a lumen defined within the detection device.

In at least one embodiment, of a single injection method to obtain a parallel tissue conductance within a luminal organ of the present disclosure, the method comprises the steps of introducing at least part of a detection device into a luminal organ at a first location, the detection device having a detector, applying current to the detection device, injecting a solution having a known conductivity into the luminal organ at or near the detector of the detection device, measuring a first output conductance indicative of the injected solution using the detector, obtaining a second output conductance indicative of a bodily fluid native to the luminal organ using the detector, and calculating a parallel tissue conductance based in part upon the first output conductance, the second output conductance, and the known conductivity of the injected solution.

In at least one embodiment of a single injection method to determine a cross-sectional area of a luminal organ of the present disclosure, the method comprises the steps of introducing at least part of a detection device into a luminal organ at a first location, the detection device having a detector, applying current to the detection device, injecting a solution having a known conductivity into the luminal organ at or near the detector of the detection device, measuring a first output conductance indicative of the injected solution using the detector, obtaining a second output conductance indicative of a bodily fluid native to the luminal organ using the detector, and calculating a cross-sectional area of the luminal organ at the first location based in part upon the first output conductance, the second output conductance, and the known conductivity of the injected solution.

In at least one embodiment of a single injection method to determine a cross-sectional area of a luminal organ, the method comprises the steps of introducing at least part of a detection device into a luminal organ at a first location, the detection device having a detector, applying current to the detection device using a stimulator, introducing a first signal having a first frequency and a second signal having a second frequency through the detection device, measuring a first output conductance of the first signal and the second signal, at the first location is connection with a fluid native to the first location, said fluid having a first conductivity, injecting a solution having a known conductivity into the luminal organ at or near the detector of the detection device, measuring a second output conductance of die first signal and the second signal at the first location is connection with the injected solution, and calculating a cross-sectional area of the luminal organ at the first location based in part upon the second output conductance and the known, conductivity of the injected solution.

In at least one embodiment of a single injection method to assess the composition of a plaque within a luminal organ, the method comprises the steps of introducing at least part of a detection device into a luminal organ at a plaque site, the detection device having a detector, applying current: to the detection device using a stimulator, introducing a first signal having a first frequency and a second signal having a second frequency through the detection device, measuring a first output conductance of the first signal and the second signal at the first location in connection with a fluid native to the first location, said fluid having a first conductivity, injecting a solution having a known conductivity into the luminal organ, at or near the detector of the detection device, measuring & second output conductance of the first signal and the second signal at the first location in connection with the injected solution, and determining plaque-type composition of a plaque at the plaque site based in part upon fee second output conductance and the known conductivity of the injected solution.

In at least one embodiment of a system to obtain a parallel tissue conductance within a luminal organ, the system comprises a detection device having a detector, and a frequency generator coupled to the detection device. In another embodiment, the detector is capable of measuring an output conductance. In yet another embodiment, the detector comprises two detection electrodes positioned in between, two excitation electrodes. In an additional embodiment, the two excitation electrodes are capable of producing an electrical field. In yet an additional embodiment, the frequency generator is capable of generating signals having at least two distinct frequencies through the detection device.

In at least one embodiment of a system to obtain a parallel tissue conductance within a luminal organ, the system further comprises a deconvolution device. In an additional embodiment, the deconvolution device is capable of deconvoluting an output conductance to obtain a first conductance value and a second conductance value from the output conductance. In yet an additional embodiment, the system, further comprises a stimulator coupled to the detection device. In another embodiment, the stimulator is capable of exciting a current to the detection device.

In at least one embodiment of a system to obtain a parallel tissue conductance within a luminal organ, the system further comprises a data acquisition and processing system coupled to the detection device. In another embodiment, the data acquisition and processing system is capable of receiving conductance data from the detector and calculate parallel tissue conductance. In yet another embodiment, the data acquisition and processing system is further capable of calculating a cross-sectional area of a luminal organ based, upon the conductance data. In an additional embodiment, the data acquisition and processing system is further capable of determining plaque-type composition of a plaque within a luminal organ based upon the conductance data.

### BRIEF DESCRIPTION OF THE DRAWINGS

FIG. 1 shows the flow of a dual frequency stimulus to obtain a dual conductance which can subsequently be deconvoluted, according to an embodiment of the present disclosure;

FIG. 2A shows an exemplary system for obtaining a parallel tissue conductance within a luminal organ according to an embodiment of the present disclosure;

FIG. 2B shows an exemplary detection device of an exemplary system for obtaining a parallel tissue conductance within a luminal organ having impedance measuring electrodes supported in front of a stenting balloon thereon, according to an embodiment of the present disclosure;

FIG. 2C shows m exemplary detection device of an exemplary system for obtaining a parallel tissue conductance within a luminal organ having impedance measuring electrodes within and in front of a balloon thereon, according to an embodiment of the present disclosure;

FIG. 2D shows an exemplary detection device of an exemplary system for obtaining a parallel tissue conductance within a luminal organ having an ultrasound transducer within and in front of a balloon thereon, according to an embodiment of the present disclosure;

FIG. 2B shows an exemplary detection device of an exemplary system for obtaining a parallel tissue conductance within a luminal organ without a stealing balloon, according to an embodiment of the present disclosure;

FIG. 2F shows an exemplary detection device of an exemplary system for obtaining a parallel tissue conductance within a luminal organ having wire and impedance electrodes, according to an embodiment of the present disclosure;

FIG. 2G shows an exemplary detection device of an exemplary system for obtaining a parallel tissue conductance within a luminal organ having multiple detection electrodes, according to an embodiment of the present disclosure;

FIGS. 2H, 2I and 2J show at least a portion of exemplary systems for obtaining a parallel tissue conductance within a luminal organ according to embodiments of the present disclosure;

FIG. 3 shows steps of an exemplary method for obtaining a parallel tissue conductance within a luminal organ using a single injection method according to an embodiment of the present disclosure;

FIG. 4 shows steps of another exemplary method for obtaining a parallel tissue conductance within, a luminal, organ using a single injection method according to an embodiment of the present disclosure;

FIG. 5A shows a balloon distension of the lumen of a coronary artery according to an embodiment of the present disclosure; and

FIG. 5B shows a balloon distension, of a stent into the lumen of a coronary artery according to an embodiment of the present disclosure,

### DETAILED DESCRIPTION

For the purposes of promoting an understanding of the principles of the present disclosure, reference will now he made to the embodiments illustrated m the drawings, and specific language will be used to describe the same. It will nevertheless be understood that no limitation of the scope of this disclosure is thereby intended.

### CSA and Gp

The present disclosure provides for systems and methods for obtaining parallel tissue conductances to, for example, measure cross-sectional areas and pressure gradients in luminal organs such as, for example, blood vessels, heart valves, and other visceral hollow organs,

A two injection method allowing for the simultaneous determination of cross-sectional area (CSA) and parallel conductance (Gₚ) of luminal organs are currently known in the art by way of U.S. Pat. No. 7,454,244 to Kassab. As referenced therein, each injection provides a known conductivity-conductance (σ-G) relation or equation as per an Ohm's law modification that accounts for parallel conductance (namely current losses from the lumen of vessel):

G=(CSA/L)σ÷Gₚ [1]

wherein G is the total conductance. CSA is the cross-sectional area of the luminal organ (which may include, hut is not limited to, various bodily lumens and vessels, including blood vessels, a biliary tract, a urethra, and an esophagus, for example), L is a constant for the length of spacing between detection electrodes of the detection device used, σ is the specific electrical conductivity of the fluid, and Gₚ is the parallel conductance (namely the effective conductance of the structure outside of the fluid).

Mathematically, two equations (corresponding to two injections) and two unknowns produce a deterministic solution for CSA and Gₚ. Normal and half-normal saline solutions, for example, are routinely used clinically and therefore, are the logical choice for varying the σ-G relation to produce two equations for the two unknowns.

In order to reduce the number of steps that a clinician must perform, it would be ideal to reduce the number of injections. The disclosure of the present application addresses the same, providing a clinician with the alternative of using a single injection instead of being required to use two injections to determine cross-sectional areas of luminal organs.

The following analysis allows a single injection of saline to provide the desired. CSA and Gₚ. The additional equations referenced below are generated through multiple stimulating frequency injections; i.e., the system performs multiple current injections at baseline (in blood) and during a single saline injection. The system then determines the response (conductance) to both frequencies which allows the calculation of CSA and Gₚ uniquely.

To facilitate these determinations, the following axioms or facts established in the art are considered: (i) the conductivity of blood, does not vary over stimulating or excitation

frequencies in the range of 2-100 kHz; (ii) muscle/vessel becomes more conductive when frequency is greater than 1.2 kHz; and (iii) saline conductivity varies as a power relation with frequency.

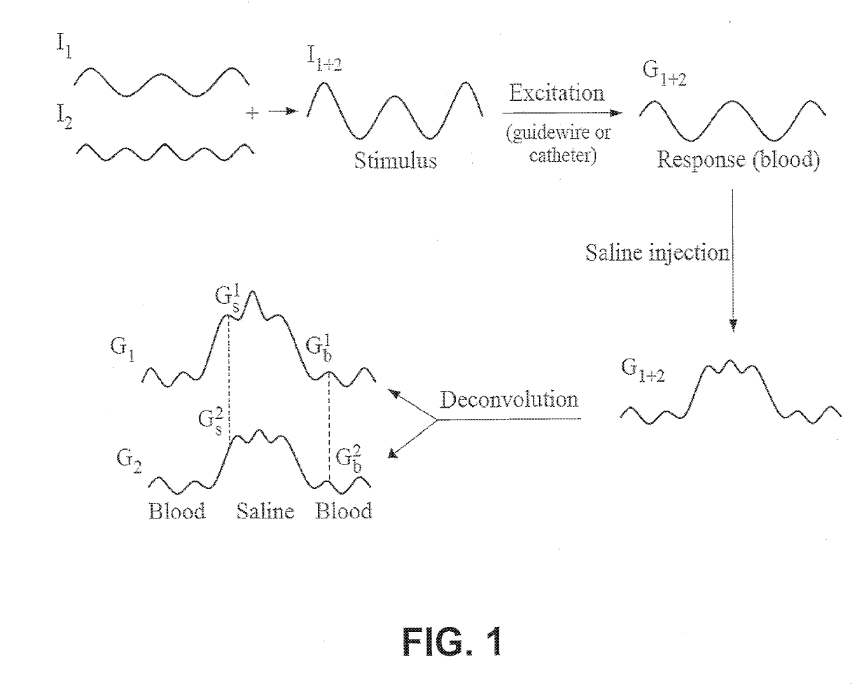

A premise of the disclosure of the present application is to stimulate with dual frequency to provide the appropriate number of equations to solve for the desired parameters (CSA and Gₚ). For example, consider a waveform of two different frequencies (e.g., 3 and 10 kHz) as the excitation frequencies as shown in FIG. 1. If those stimulating frequencies are applied to Equation [1], one will obtain the following;

In blood (b):

G¹_{b}=(CSA/L) σ_{b}+G¹ₚ [2]

and

G²ₛ=(CSA/L) σ_{b}+G¹ₚ [3]

where 1 and 2 correspond to the two different frequencies, respectively; and

### During Saline(s) Injection:

G¹ₛ=(CSA/L) σ¹ₛ+G¹ₚ [4]

and

G²ₛ=(CSA/L) σ²ₛ+G²ₚ [5]

The only assumption applicable to the foregoing is that the parallel conductance (Gₚ) is the same with blood or blood which, is physically reasonable and has been proven for the heart muscle. As referenced above, L is known from the device design (guidewire or catheter, for example), σ¹ₛ and σ²ₛ represent calibration constants measured for the device, and G¹_{b}, G²_{b}, G¹ₛ, and G²ₛ are measured for baseline blood and during the saline injection.

Therefore, there are four remaining unknowns: CSA, G¹ₚ, G²ₚ, and σ_{b}. Since there are four applicable equations (Equations [2-5]), the problem is therefore mathematically well posed and deterministic. If the change of parallel conductance (Gₚ) with frequency is relatively small, then Equations [2] and [3] become unnecessary and Equations [4] and [5] reduce to:

G¹ₛ=(CSA/L) σ¹ₛ+Gₚ [6]

and

G²ₛ=(CSA/L) σ²ₛ+Gₚ [7]

which becomes analogous to the two saline injections but with one saline injection at two different frequencies.

In general, four equations can be set up as a matrix of the form Ax=b:

[1/L 0 1 0 ] [CSAσ_{b}] [G_{b}¹]

[1/L 0 0 1] [CSA] [G_{b}²]

[0 σ¹ₛ/L 0 1] [Gₚ¹][Gₛ¹]

[0 σ²ₛ/L 0 1] [Gₚ²] [Gₛ²]

wherein A is the 4×4 matrix of known quantities, x is the 1×4 matrix of unknown quantities (CSA, σ_{b}, G¹, Gₚ²), and b is the 1×4 matrix of known quantities,

A single injection method may also be utilized in accordance with the following, whereby the desired CSA and Gp can be obtained with two equations, one stemming from a fluid injection (such as saline), and the other stemming from measured blood conductivity.

Using such an exemplary embodiment of a single injection method, and as referenced generally above, blood conductivity can be measured for each patient by recording the electrical conductance within the device (such as an introducer catheter, for example) with known dimensions. Ohm's law can then be used in the catheter, wherein Gp=0, as follows:

G=(CSA/L) σ_{b} [8]

Since G can be measured within the catheter (which h then already inserted in the body of the patient) having a known diameter or CSA, and since L (the distance between detection electrodes) is also a known parameter, σ_{b} (the conductivity of blood) can determined for each patient prior to advancing the device to the site of interest for sizing measurements. Some example measurements obtained during swine testing provided values that range from 0.827-0.899 (with average of 0.866 in appropriate units) in one animal and values that range from 0.871-0.889 (with average of 0.866) in another animal. These compare to mean values of 0.694 and 1.362 for 0.45% and 0.9% NaCl (in the same units), respectively. Blood conductivity is intermediate to normal and half normal saline.

With the average σ_{b} known. Equation [1] can then be rewritten as;

Gₛ=(CSA/L) σₛ+Gₚ [9]

and

G_{b}=(CSA/L) σ_{b}+G²ₚ [10]

wherein Gₛ and G_{b} correspond to electrical conductance measurements in the presence of saline (s) and blood (b), respectively. These solution to such a 2×2 matrix is then identified as

$$\begin{matrix}CSA ( t ) = L \frac{[ {G}_{s} ( t ) - {G}_{b} ( t ) ]}{[ {σ}_{s} - {σ}_{b} ]} \\ and & [ 11 ] \\ {G}_{p} ( t ) = \frac{[ {σ}_{s} \cdot {G}_{b} ( t ) - {σ}_{b} \cdot {G}_{s} ( t ) ]}{[ {σ}_{s} - {σ}_{b} ]} & [ 12 ]\end{matrix}$$

Experimental measurements in swine using the two injection method as referenced above compared to present one injection method compare very well, within accepted error tolerance. For example, studies using said one injection method resulted in an obtained mean value of 5.7±0.22 mm (from several blood vessel measurements that ranged from 5.53 to 5.95 mm), and 5.2±0.22 mm (from the same measurements that ranged from 5.01 to 5.41 mm for three respective blood vessel measurements) using the aforementioned two injection method. The actual blood vessel measurement was 5.4 mm, and both methods were within 5% of the actual measurement.

In at least one embodiment of a single injection method of the present disclosure, the injection includes adenosine. Adenosine, used in said method, can also provide hyperemic velocity measurements to determine coronary flow reserve and in turn fractional flow reserve as previously outlined.

The present single injection method has a number of significant and non-obvious differences as compared to prior two injection methods. Instead of using 0.45% NaCl (or some other known salinity or fluid conductivity), the present single injection method uses the patient's own blood with patient-specific blood conductivity as determined in the catheter in vivo prior to measurement. In addition, a single saline injection containing adenosine that provides the sizing also provides the hyperemia velocity measurements as referenced herein.

The present disclosure allows for accurate measurements of the luminal cross-sectional area of organ stenosis within acceptable limits to enable accurate and scientific stent sizing and placement in order to improve clinical outcomes by avoiding under or over deployment and under or over sizing of a stent which can cause acute closure, or in-stent re-stenosis. In an exemplary embodiment, an angioplasty or stent balloon positioned upon fee device (catheter or wire, for example) includes impedance electrodes supported by the catheter in front of the balloon. These electrodes enable the immediate measurement of the cross-sectional area of the vessel during the balloon advancement, providing a direct measurement of non-stenosed area and allowing the selection of the appropriate stent size, in one approach, error due to the loss of current in the wall of the organ and surrounding tissue is corrected by injection of a saline solutions or other solutions with a known conductivities. In at least one embodiment, impedance electrodes are located in the center of the balloon, in order to deploy the stent to the desired cross-sectional area. These embodiments and procedures substantially improve the accuracy of stenting and the outcome and reduce the cost.

Other embodiments make diagnosis of valve stenosis more accurate and more scientific by providing a direct accurate measurement of cross-sectional area of a valve annnlus, independent of the flow conditions through the valve. Other embodiments improve evaluation of cross-sectional area and flow in organs like the gastrointestinal tract and the urinary tract.

Embodiments of the present disclosure overcome the problems associated with determination of the size (cross-sectional area) of luminal organs, such as, for example, in the coronary arteries, carotid, femoral, renal and iliac arteries, aorta, gastrointestinal tract, urethra and ureter. Exemplary embodiments also provide methods for registration of acute changes in wall conductance, such as, for example, due to edema or acute damage to the tissue, and for detection of muscle spasms/contractions.

As referenced herein, and in at least one exemplary embodiment, there is provided an angioplasty catheter with impedance electrodes near the distal end of the catheter (in front of the balloon, for example) for immediate measurement of the cross-sectional area of a vessel lumen during balloon advancement Such a catheter would include electrodes for accurate detection of organ luminal cross-sectional area and ports for pressure gradient measurements. Hence, it is not necessary to change catheters such as with the current use of intravascular ultrasound.

In an exemplary embodiment, such a catheter provides direct measurement of the non-stenosed area, thereby allowing the selection, of an appropriately sized stent. In another embodiment, additional impedance electrodes may be incorporated in the center of the balloon on the catheter in order to deploy the stent to the desired cross-sectional area. The procedures described herein substantially improve the accuracy of stealing and improve the cost and outcome as well.

In another exemplary embodiment, the impedance electrodes are embedded within a catheter to measure the valve area, directly and independent of cardiac output or pressure drop mid therefore minimize errors in the measurement of valve area. As such, measurements of area are direct and not based on calculations with underlying assumptions. In another exemplary embodiment, pressure sensors can be mounted proximal and distal to the impedance electrodes to provide simultaneous pressure gradient recording.

### Plaque-type and Gp

The disclosure of the present application further provides systems and methods for determining the type and/or composition of a plaque that may be engaged within a blood vessel, permitting accurate and reproducible measurements of the type or composition of plaques is blood vessels within acceptable limits. The understanding of a plaque type or composition allows a health care professional to better assess the risks of the plaque dislodging from its position and promoting infarct downstream. For example, the disclosure of the present application enables the determination of a plaque type and/or composition in order to improve patient health by allowing early treatment options for undersized (but potentially dangerous) plaques that could dislodge and cause infarcts or other health problems. As discussed above, such determination of plaque information allows for removal or other disintegration of a smaller plaque that may otherwise not be of concern under conventional thought merely because of its smaller size. However, smaller plaques, depending on their composition, are potentially lethal, and the disclosure of the present application serves to decrease the ill effects of such plaques by assessing their type and composition when they are still “too small” to be of concern for standard medical diagnoses.

Gₚ is a measure of electrical conductivity through the tissue and is the inverse of electrical resistivity. Fat or lipids have a higher resistivity to electrical flow or a lower Gₚ than compared to most other issues. For example, lipids have approximately ten times (10×) higher resistivity or ten times (10×) lower conductivity than vascular tissue. In terms of conductivities, fat has a 0.023 S/m value, blood vessel wall has 0.32 S/m, and blood has a 0.7 S/m. Because unstable plaques are characterized by a higher lipid core, at least one purpose of the disclosure of the present application is to allow a clinician, for example, to use the value of Gₚ to identify vulnerable plaque.

Studies indicate that Gₚ is about 70-80% for a normal vessel. This value is significantly reduced when lipid is present in the vessel, wall. In other words, the lipid insulates the vessel and significantly reduces the current loss through the wall. The degree of reduction of Gₚ will be dependent on the fraction of lipid in the plaque. The higher the fraction of lipid, the smaller the value of Gₚ, and consequently the greater the risk of plaque rupture which can cause acute coronary syndrome. Thus, the exemplary embodiments described throughout this disclosure are used to develop a measure for the conductance, Gₚ, which in turn is used as a determinant of the type and/or composition of the plaque in the region of measurement.

In an exemplary embodiment, the data on parallel conductance as a function of longitudinal position along the vessel can be exported from an electronic spreadsheet, such as, for example, a Microsoft Excel file, to a diagramming software, such as AutoCAD, where the software uses the coordinates to render the axial variation of Gₚ score (%Gₚ).

Furthermore, the Gₚ score may be scaled through a scaling model index to simplify its relay of information to a user. An example of a scaling index used in the present disclosure is to designate a single digit whole number to represent the calculated conductance Gₚ. In such a scaling index, for example, “0” would designated a calculated Gₚ of 0-9%; “1” would designate a calculated Gₚ of 10-19%; “2” would designate a calculated Gₚ of 20-29%; . . . ; and “9” would designate a calculated Gₚ of 90-100%. In this sealing index example, a designation of 0, 1, 2, 3, 4, 5 or 6 would represent a risky plaque composition, with the level of risk decreasing as the scaling number increases, because the generally low level of conductance meaning generally higher fat or lipid concentrations. In contrasts, a designation of 7, 8 or 9 would generally represent a non-risky plaque composition, with the level of risk decreasing as the scaling number increases, because the generally higher level of conductance meaning generally lower fat or lipid concentrations.

For example, for a given determination of a conductance value of 68%, the resultant plaque type would be deemed as “6” or somewhat fatty. This would be a simple automated analysis of the plaque site under consideration based on the teachings and discoveries of the present disclosure as described throughout this disclosure. Of course, the range for die scaling model described above could be pre-set by the manufacturer according to established studies. but may be later changed by the individual clinic or user based on further or subsequent studies.

Gₚ and other relevant measures such as distensibility, tension, etc. may then appear on a computer screen, and the user can then remove the stenosis by distension or by placement of a stent. The value of Gₚ, which reflects the “hardness” (high Gₚ) or “softness” (low Gₚ), can be used in selection of high or low pressure balloons as known in the arts.

Regarding plaque-type determination, using two different frequencies (3 kHz and 10 kHz, for example), solving the above-referenced matrix provides for a ratio of parallel conductance at the two frequencies to assess plaque-type. Regarding the matrix, the solutions of unknown quantities can be provided as follows:

σ_{b}=[L (G_{b}²+((Gₛ² σ¹ₛ−G¹ₛ σ²ₛ)/(σ²ₛ−σ¹ₛ))]/CSA [13]

CSA=L (Gₛ¹−G²ₛ)/(σ¹ₛ−σ²ₛ) [14]

Gₚ¹=(G_{b}¹−G_{b}²)−((Gₛ² σ¹ₛ−G¹ₛ σ²ₛ)/(σ¹ₛ−σ²ₛ)) [15]

Gₚ²=(Gₛ² σ¹ₛ σ¹ₛ−G¹ₛ σ²ₛ)/(σ¹ₛ−σ²ₛ) [16]

The ratio of parallel, conductance at the two different frequency is given by;

[Gₚ²]/[Gₚ¹]=(Gₛ² σ¹ₛ−G¹ₛ σ²ₛ)/((G_{b}¹−G_{b}²+Gₛ²) σ¹ₛ−(G_{b}¹−G_{b}²+Gₛ¹) σ²ₛ) [17]

This ratio (Equation [17]) can be used to assess plaque composition. In a normal vessel, the ratio of parallel conductance at two frequencies (3 kHz and 10 kHz, for example) is 4.8 or roughly 5. If the vessel was entirely surrounded by fat (a lipid lesion), the ratio would reduce to 1.03 or roughly 1. Hence, the ratio of parallel, conductance at the two frequencies can fee used as an index of lipid composition where 1 (completely lipid) and 5 (no lipid) similar to previous scale referenced herein. In summary, the first sale referenced above shows that a reduction of parallel conductance at any given frequency implies the presence of lipid to different extant, and this second scale considers the dependence of parallel conductance on frequency (with almost constant or no change with frequency suggesting high lipid composition), providing two orthogonal parameters to characterize the lesion composition.

In use, an exemplary system of the present disclosure provides a user with an effective and powerful tool to relay information about a vessel site and any plaque housed therein. A user could first consider the CSA level as an exemplary device is pulled through the site or as numerous electrodes calculate the CSA as their designated cross-sectional place, as described generally herein. If there is little to no changes in the CSA value, then the user could acknowledge that there is little to no obstructions or plaques within the lumen of the blood vessel. However, if there is some change in the value of the CSA, then the conductance measurement and plaque type information could be monitored to determine the extent to which plaque formation is present as well as the type of plaque, as determined by the seating model whole number displayed, as described herein.

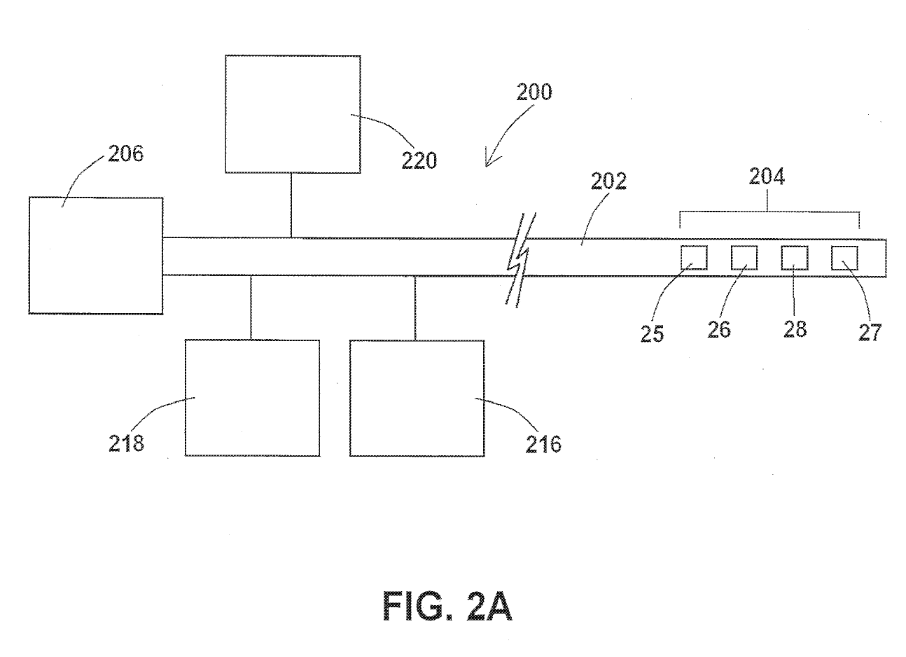

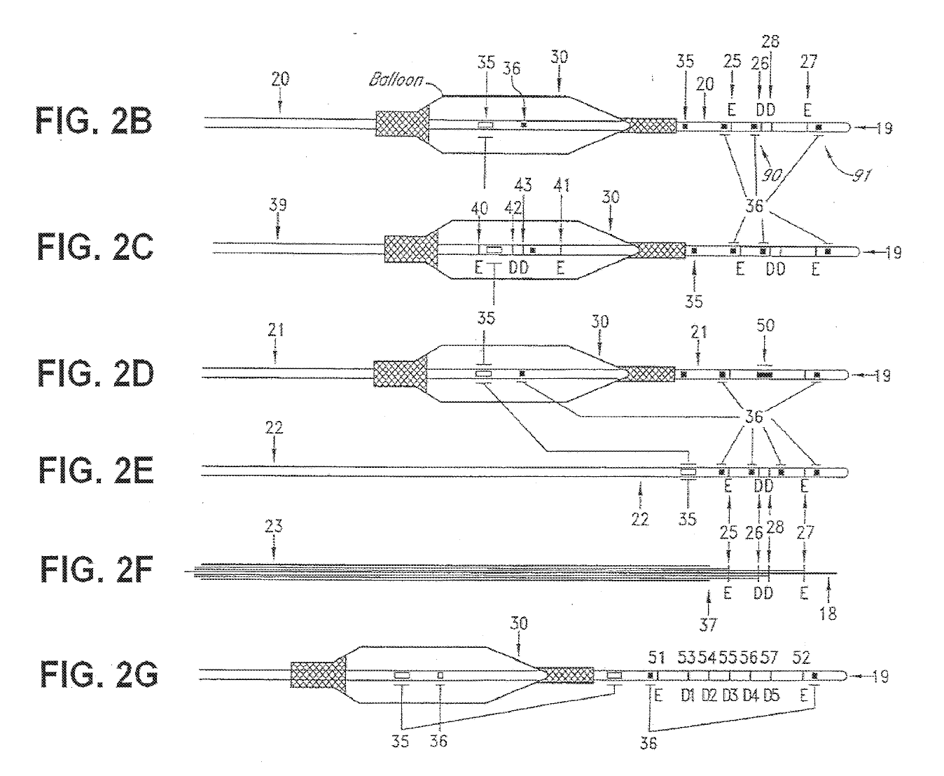

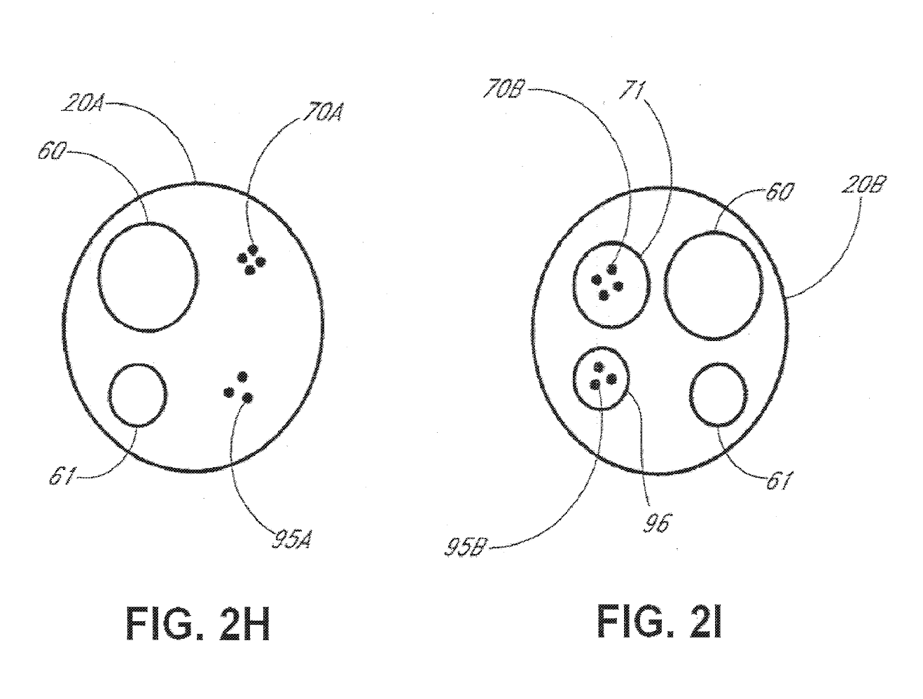

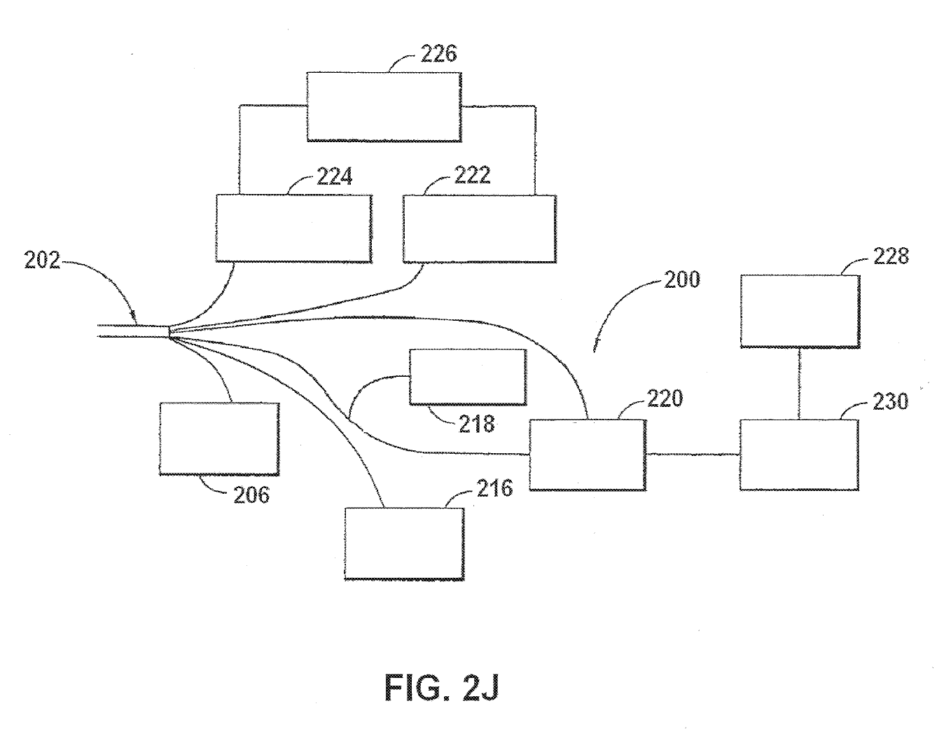

Reference will now be made to the various systems and methods of the present disclosure as shown in the figures. FIG. 1 shows a schematic for using signals having differing frequencies in accordance with the present disclosure to allow for the calculation of CSA within a luminal organ. As shown in FIG. 1, two input signals having different frequencies I₁ and I2) are combined to form one combined stimulating signal (I₁₊₂). When the combined stimulating signal flows through, for example, a detection device 202 (as referenced below in FIG. 2A), an output conductance (G₁₊₂) in response to said stimulating signal may be obtained. Such an output conductance, absent of any solution injection, would be indicative of the conductance of the fluid native to the area (blood, for example). If such a signal flows through the device during the time of a saline injection, for example, the output conductance would foe indicative of the saline solution.

Such an output (of dual conductances) can lead to the following. The b matrix values are shown in FIG. 1 for blood and saline and can be determined accordingly. Once A and b are inputted, x can be solved in conventional way to determine the CSA and parallel conductance (Gₚ). As shown in FIG. 1, the combined response can be deconvoluted to produce the desired parameters to calculate the CSA and parallel conductance simultaneously.

An exemplary system for obtaining a parallel tissue conductance within a luminal organ of the present disclosure is shown in FIG. 2A. As shown, in FIG. 2A, an exemplary embodiment of a system 200 of the present disclosure comprises a detection device 202 having a detector 204, and a frequency generator 206 coupled to detection device 202. Frequency generator 206, in at least one embodiment, is capable of generating signals having at least two distinct frequencies through defection device 202. An exemplary frequency generator 206 may include, but is not limited to, an arbitrary waveform generator or two signal generators. In at least one embodiment of an arbitrary waveform generator, the output conductance can be filtered at the appropriate frequency to derive the desired conductance for each frequency. In at least one embodiment of system 200 of the present disclosure, detector 204 comprises detection electrodes 26, 28 positioned in between excitation electrodes 25, 27, wherein excitation, electrodes 25, 27 are capable of producing an electrical field,

In an exemplary embodiment of system 200, system 200 further comprises a deconvolution device 216, whereby deconvolution device 216 is capable of filtering an output conductance to obtain a first conductance value and a second conductance value from the output conductance, and/or whereby deconvolution device 216 is capable of filtering an output frequency to obtain a first resulting frequency and a second resulting frequency from the output frequency. Deconvolution device 216 may be coupled to any number of elements of system 200, including, but not limited to, detection device 202, detector 204, and/or frequency generator 206. In the exemplary embodiment of system 200 shown in FIG. 2A, deconvolution device is shown as being coupled to detection device 202,

Furthermore, and in an exemplary embodiment of a system 200 of the present disclosure, system 200 may further comprise a stimulator 218 capable of applying/exciting a current to detection device 202. An exemplary system 200 of the present disclosure may also comprise a data acquisition and processing system 220 capable of receiving conductance data from detector 204 and calculating parallel tissue conductance. In various embodiments of data acquisition and processing systems 220, data, acquisition and processing systems 220 may be further capable of calculating a cross-sectional area of a luminal organ and/or determining plaque-type composition of a plaque within a luminal organ, based upon the conductance data.

In addition, an exemplary detection device 202 of the present disclosure may comprise any number of devices 202 as shown in FIGS. 2B-2G. Referring to FIGS. 2B, 2C, 2D, and 2E, several exemplary embodiments of the detection devices 202 are illustrated. The detection devices 202 shown contain, to a varying degree, different electrodes, number and optional balloon(s). With reference to the embodiment shown in FIG. 2B, there is shown an impedance catheter 20 (an exemplary detection device 202) with four electrodes 25, 26, 27 and 28 placed close to the tip 19 of the catheter 20. Proximal to these electrodes is an angiography or stenting balloon 30 capable of being used for treating stenosis. Electrodes 25 and 27 are excitation electrodes, while electrodes 26 and 28 are detection electrodes, which allow measurement of cross-sectional area, during advancement of detection device 202, as described in further detail below. The portion of catheter 20 within balloon 30 includes an infusion port 35 and a pressure port 36.

Catheter 20 may also advantageously include several miniature pressure transducers (not shown) carried by the catheter or pressure ports for determining the pressure gradient proximal at the site where the CSA is measured. The pressure may be measured inside the balloon and proximal, distal to and at the location of the cross-sectional area measurement, and locations proximal and distal thereto, thereby enabling the measurement of pressure recordings at the site of stenosis and also the measurement of pressure-difference along or near the stenosis. In at least one embodiment and as shown in FIG. 2B, catheter 20 includes pressure port 99 and pressure port 91 proximal to or at the site of the cross-sectional measurement for evaluation of pressure gradients. As described below with reference to FIGS. 2H, 2I, and 2J, and in at least one embodiment, pressure ports 90, 91 are connected by respective conduits in catheter 20 to pressure sensors within system 200. Such pressure sensors are well known in the art and include, for example, fiber-optic systems, miniature strain gauges, and perfused low-compliance manometry.

In at least one embodiment, a fluid-filled silastic pressure-monitoring catheter is connected to a pressure transducer. Luminal pressure can be monitored by a low compliance external pressure transducer coupled to the infusion channel of the catheter. Pressure transducer calibration may be carried out by applying 0 and 100 mmHg of pressure by means of a hydrostatic column, for example.

In an exemplary embodiment, and shown in FIG. 2C, catheter 39 includes another set of excitation electrodes 40, 41 and detection electrodes 42, 43 located inside the angioplastic or staling balloon 30 for accurate determination of the balloon cross-sectional area during angioplasty or stent deployment These electrodes are in addition to electrodes 25, 26, 27 and 28.

In another exemplary embodiment, and as shown, in FIG. 2G, several cross-sectional areas can be measured using an array of 5 or more electrodes. Here, the excitation electrodes 51, 52, are used to generate the current while detection electrodes 53, 54, 55, 56 and 57 are used to detect the current at their respective sites.

The tip of an exemplary catheter can be straight, curved or with an angle to facilitate insertion into the coronary arteries or other lumens, such as, for example, the biliary tract. The distance between the balloon and the electrodes is usually small, m the 0.5-2 cm range, but can he closer or further away, depending on the particular application or treatment involved.

In at least another embodiment, and shown in FIG. 2D, catheter 21 has one or more imaging or recording device, such as, for example, ultrasound transducers 50 for cross-sectional area and wall thickness measurements. As shown in this exemplary embodiment transducers 50 are located near the distal lip 19 of catheter 21.

FIG. 2E shows an exemplary embodiment of an impedance catheter 22 without an angioplastic or stealing balloon. This catheter 22 also comprises an infusion or injection port 35 located proximal relative to the excitation electrode 25 and pressure port 36,

With reference to the exemplary embodiment shown in FIG. 2F, electrodes 25, 26, 27, 28 can also be built onto a wire 18, such as, for example, a pressure wire, and inserted through a guide catheter 23 where the infusion of bolus can be made through the lumen of the guide catheter 37. Various wire 18 embodiments can be used separately (i.e., without a catheter), or can be used in connection with a guide catheter 37 as shown in FIG. 2B,

With reference to the embodiments shown in FIGS. 2B-2G, the impedance catheter advantageously includes optional ports 35, 36, 37 for suction of contents of the organ or infusion of fluid. Suction/infusion ports 35, 36, 37 can be placed as shown with the balloon or elsewhere both proximal or distal to the balloon on the various catheters. The fluid inside the balloon may be any biologically compatible conducting fluid. The fluid to inject through the infusion port or ports can be any biologically compatible fluid but the conductivity of the fluid is selected to be different from that of blood (e.g., saline).

In at least another embodiment (not illustrated), an exemplary catheter contains m extra channel for insertion of a guide wire to stiffen the flexible catheter during the insertion or data recording. In yet another embodiment (not illustrated), the catheter includes a sensor for measurement of the flow of fluid in the body organ.

As described below with reference to FIGS. 2H, 2I, and 2J, the excitation and detection electrodes are electrically connected to electrically conductive leads in the catheter for connecting the electrodes to the stimulator 218, for example.

FIGS. 2H and 2I illustrate two exemplary embodiments 20A and 20B of the catheter in cross-section. Bach embodiment has a lumen 60 for inflating and deflating a balloon and a lumen 61 for suction and infusion. The sizes of these lumens can vary in size. The impedance electrode electrical leads 70A are embedded in the material of the catheter to the embodiment in FIG. 2H, whereas the electrode electrical, leads 70B are tunneled through a lumen 71 formed within the body of catheter 708 in FIG. 2I.

Pressure conduits for perfusion manometry connect the pressure ports 90, 91 to transducers included in system 200. As shown, in FIG. 2H, pressure conduits 95A may be formed, in 20A. In another exemplary embodiment, shown in FIG. 2I, pressure conduits 95B constitute individual conduits within a teasel 96 formed in catheter 208. In tee embodiment described above where miniature pressure transducers are carried by the catheter, electrical conductors will be substituted tor these pressure conduits.

At least a portion of an exemplary system for obtaining a parallel tissue conductance within a luminal organ of the present disclosure is shown in FIG. 2J. As shown in FIG. 2J, an exemplary system 200 of the present disclosure comprises a detection device operably connected to a manual or automatic system 222 for distension of a balloon and to a system 224 for infusion of fluid or suction of blood. The fluid, in an exemplary embodiment, may be heated to 37-39° C. or equivalent to body temperature with testing unit 226. In addition, and as shown in FIG. 23, system 200 may comprise a stimulator 218 to provide a current to excite detection device 202, and a data acquisition and processing system 220 to process conductance data. Furthermore, an exemplary system 200 may also comprise a signal amplifier/conditioner (not shown) and a computer 228 for additional data processing as desired. Such a system 200 may also optionally contain signal conditioning equipment for recording of fluid flow in the organ.

In an exemplary embodiment the system 200 is pre-calibrated and the detection device 202 is available in a package. In such an embodiment, for example, the package may also contains sterile syringes with the fluid(s) to be injected. The syringes, in an exemplary embodiment, may be attached to heating unit 226, and after heating of the fluid by heating unit 226 and placement of at least part of detection device 202 in the luminal organ of interest, the user presses a button that initiates the injection with subsequent computation of the desired parameters. The parallel conductance, CSA, plaque-type, and other relevant measures such as distensibility, tension, etc., may then typically appear on the display of computer 228. In such an embodiment, the user can then remove the stenosis by distension or by placement of a stent

If more than one CSA is measured, for example, system 200 can also contain a multiplexer unit or a switch between CSA channels. In at least one embodiment, each CSA measurement will be through separate amplifier units. The same may account for the pressure channels as well.

In at least one embodiment, the impedance and pressure data are analog signals which are converted by analog-to-digital converters 230 and transmitted to a computer 228 for on-line display, on-line analysis and storage. In another embodiment, all data handling is done on an entirely analog basis. The analysis may also includes software programs for reducing the error due to conductance of current in fee organ wall and surrounding, tissue and for displaying the 2D or 3D-geometry of the CSA distribution along the length of the vessel along with the pressure gradient. In an exemplary embodiment of the software, a finite element approach or a finite difference approach is used to derive the CSA of the organ stenosis taking parameters such as conductivities of the fluid in the organ and of the organ wall and surrounding tissue into consideration. In another embodiment, the software contains the code for reducing the error in luminal CSA measurement by analyzing signals during interventions such as infusion of a fluid into the organ or by changing the amplitude or frequency of the current from the constant current amplifier. The software chosen for a particular application preferably allows computation of the CSA with only a small error instantly or within acceptable time during the medical procedure.

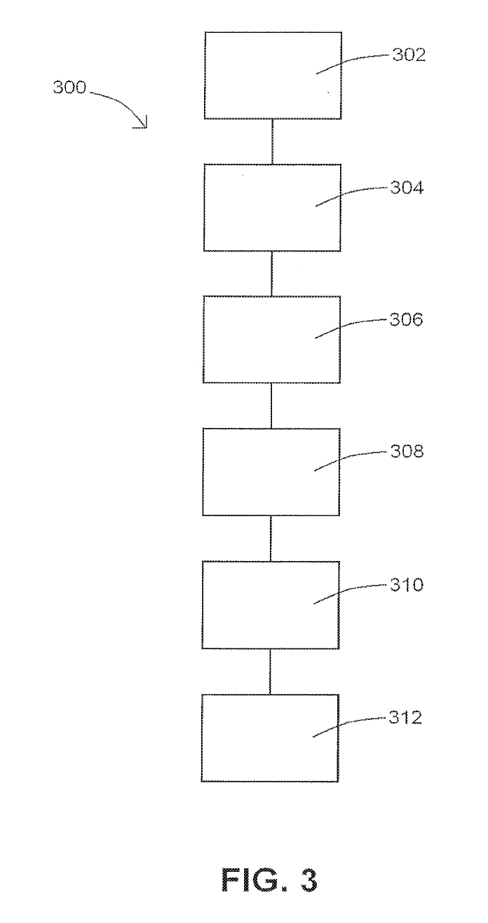

Steps of an exemplary single injection method of the present disclosure are shown in FIG. 3. As shown in FIG. 3, an exemplary method 300 comprises the step of introducing at least part of a detection device 202 into a luminal organ at a first location (introduction step 302), whereby detection device 202 comprises a detector 204, and applying current to detection device 202 to allow detector 204 to operate (current application step 304). The application/excitation of current may be performed using a stimulator 218. Method 300, in at least one embodiment further comprises the steps of introducing a first signal having a first frequency and a second signal having a second frequency through detection device 202 (frequency introduction step 306), and injecting a solution having a known conductivity into the luminal organ at or near detector 204 of detection device 202 (solution injection step 308). to an exemplary embodiment of a method 300 of the present disclosure, frequency introduction step 306 is performed using a frequency generator 206.

After injection of the solution exemplary method 300 further comprises the step of measuring an output conductance of the first signal and the second signal at the first location (conductance measurement step 310). and the step of calculating a parallel tissue conductance at the first location (calculation step 312), in an exemplary embodiment, based in part upon the output conductance and the conductivity of the injected solution.

Calculation step 312, in at least one embodiment, may comprise the step of calculating a cross-sectional area of the luminal organ at the first location, in an exemplary embodiment wherein the first location comprises a plaque site, calculation step 312 may comprise the step of determining plaque-type composition of a plaque at the plaque site.

Conductance measurement step 310 may include the measurement of an output conductance whereby the output conductance comprises a first conductance value and a second conductance value. In at least one embodiment, the first conductance value corresponds to the first frequency and the second conductance value corresponds to the second frequency. In an exemplary embodiment, calculation step 312 may comprise the step of deconvoluting the output conductance to obtain a first conductance value and a second conductance value from the output conductance. In at least one embodiment, the step of deconvoluting the output conductance is performed using a deconvolution device 216,

In at least one embodiment of a method 300 of the present disclosure, the output conductance comprises a mixed signal. In such an embodiment, calculation step 312 may further comprise the step of deconvoluting the mixed signal to obtain, a first conductance value and a second conductance value from the mixed signal.

Frequency introduction step 306 may involve the introduction of signals having frequencies with, various characteristics. For example, and in at least one embodiment, the first signal and the second signal may be repeatedly alternated to form a multiplexed signal. The alternated signals may then be separated in time by a short amount of time, for example 1 to 1000 milliseconds. In an exemplary embodiment the first signal and the second signal are separated in time by less than 100 milliseconds. In another exemplary embodiment, the first signal and the second signal are separated in time by less than 10 milliseconds. Frequency introduction step 306 may also involve the introduction of signals whereby the fest signal and the second signal are combined to form a combined signal.

In an exemplary embodiment of conductance measurement step 310 of an exemplary method 300 of the present, disclosure, conductance measurement step 310 may be performed using an exemplary detection device 202. In at least one embodiment of a detection device 202 used in connection with a method 300 of the present disclosure, detector 204 of detection device 202 comprises detection electrodes 26, 28 positioned in between excitation electrodes 25, 27, wherein excitation electrodes 25, 27 are capable of producing an electrical field.

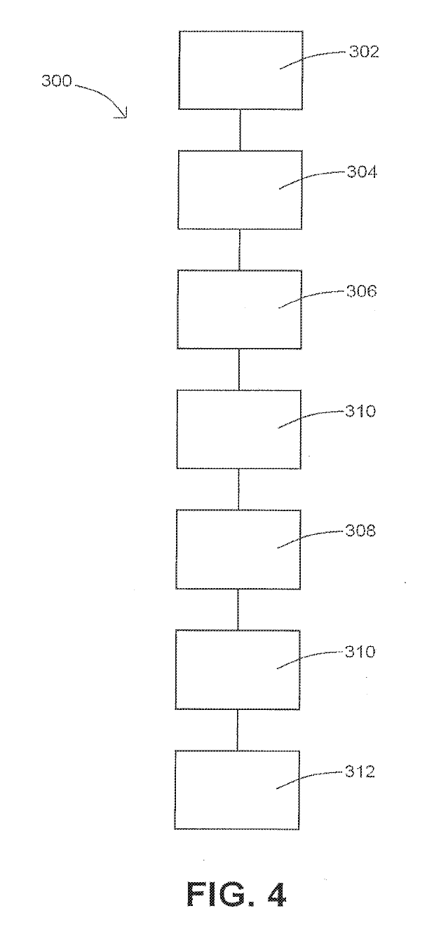

In at least another exemplary embodiment of a method 300 of the present disclosure, and as shown in FIG. 4, method 300 comprises introduction step 302, current application step 304, and frequency introduction step 306 as referenced above. This additional exemplary method 300 then comprises the step of measuring an output conductance of a first signal and a second signal at the first location (conductance measurement step 310), whereby conductance measurement step 310 involves, in such an embodiment, measuring a first output conductance at fee first location within a luminal organ in connection with a fluid native to the first location, with the native fluid having a first conductivity. After the foregoing conductance measurement step 310 has been performed, solution injection step 308 may then be performed, followed by a second conductance measurement step 310, whereby the second conductance measurement step 310 measures a second output conductance of the first signal and the second signal at the first location in connection with the injected solution. With this acquired information, art exemplary method 300 of the present disclosure may include the step of calculating a parallel tissue conductance at the first location (calculation step 312), in such an exemplary embodiment, based in part upon the second output conductance and the known conductivity of the injected solution. Calculation step 312, in at least one embodiment, may also be performed, for example, based in part upon the first output conductance and the native conductivity of the native fluid in addition to the second output conductance and the known conductivity of the injected solution.

Various characteristics of the aforementioned signals, generating the same, conductance values, filtering, frequencies, output signals, etc., apply to any number of methods 300 referenced herein. For example, and as shown, in FIG. 4, calculation step 312 of method 300 may comprise the step of deconvoluting the second output conductance to obtain a first resulting conductance value and a second resulting conductance value from the second output conductance as referenced above in connection with method 300 shown in FIG. 3.

In addition, calculation step 312, in at least one embodiment, may comprise the step of calculating a cross-sectional area of the luminal organ, at foe first location. In an exemplary embodiment wherein the first location comprises a plaque site, calculation step 312 may comprise the step of determining plaque-type composition of a plaque at the plaque site.

To consider a method of measuring Gₚ and related impedance, which are used to determine CSA or evaluate the type and/or composition of a plaque, a number of approaches may be used. In one approach, luminal cross-sectional area, is measured by introducing a catheter from an exteriorly accessible opening (e.g., mouth, nose or anus for GI applications; or e.g., mouth or nose for airway applications) into the hollow system or targeted luminal organ. In an exemplary approach, Gₚ is measured by introducing a catheter from an exteriorly accessible opening into the hollow system or targeted luminal organ. For cardiovascular applications, the catheter can be inserted into the organs in various ways, for example, similar to conventional angioplasty. In at least one embodiment, an 18 gauge needle is inserted into the femoral artery followed by an introducer, and a guide wire is then inserted into the introducer and advanced into the lumen of the femoral artery. A 4 or 5 Fr conductance catheter is then inserted into the femoral artery via wire and the wire is subsequently retracted. The catheter tip containing the conductance (excitation) electrodes can then be advanced to the region of interest by use of x-ray (using fluoroscopy, for example). In another approach, this methodology is used on small to medium size vessels, such as femoral, coronary, carotid, and iliac arteries, for example.

With respect to the solution injection, studies indicate that an infusion rate of approximately 1 ml/s for a five second interval is sufficient to displace the blood volume and results in a local pressure increase of less than 10 mmHg in the coronary artery. This pressure change depends on the injection rate, which should be comparable to the organ flow rate. In at least one approach, dextran, albumin or another large molecular weight molecule can be added to the solution (saline, for example) to maintain the colloid osmotic pressure of the solution to reduce or prevent fluid or ion exchange through the vessel wall.

In at least one approach, the saline solution is heated to body temperature prior to injection since the conductivity of current is temperature dependent. In another approach, the injected bolus is at room temperature, but a temperature correction is made since the conductivity is related to temperature in a linear fashion.

In an exemplary approach, a sheath is inserted either through the femoral or carotid artery in the direction of flow. To access the left anterior descending (LAD) artery, the sheath is inserted through the ascending aorta. For the carotid artery, where the diameter is typically on the order of 5.0-5.5 mm, a catheter having a diameter of 1.9 mm can be used. For the femoral and coronary arteries, where the diameter is typically in the range from 3.5-4.0 mm, a catheter of about 0.8 mm diameter would be appropriate. Such a device can be inserted into the femoral, carotid or LAD artery through a sheath appropriate for the particular treatment. Measurements for all three vessels can be similarly made.

The saline solution can be injected by hand or by using a mechanical injector to momentarily displace the entire volume of blood or bodily fluid in the vessel segment of interest The pressure generated by the injection will not only displace the blood in the antegrade direction (in the direction of blood flow) but also in the retrograde direction (momentarily push the blood, backwards). In other visceral organs that may be normally collapsed, the saline solution will not displace blood as in the vessels but will merely open the organs and create a flow of the fluid.

The injection described above may be repeated at least once to reduce errors associated with, the administration of the injection, such as, for example, where the injection does not completely displace the blood or where there is significant mixing with blood. Bifurcations) (with branching angle near 90 degrees) near the -targeted luminal organ may potentially cause an error in the calculated Gₚ. Hence, generally the detection device should be slightly retracted or advanced and the measurement repeated. An additional application with multiple detection electrodes or a poll hack or push forward during injection could accomplish the same goal. Here, an army of detection electrodes can be used to minimize or eliminate errors that would result from bifurcations or branching in the measurement or treatment site.

In an exemplary approach, error due to the eccentric position of the electrode or other imaging device can be reduced by inflation of a balloon on the device. The inflation of the balloon during measurement will, place the electrodes or other imaging device in the center of the vessel away from the wall. In the case of impedance electrodes, the inflation of the balloon can be synchronized with the injection of bolus where the balloon inflation would immediately precede the bolus injection.

CSAs calculated in connection with the foregoing correspond to the area of the vessel or organ, external to the device used (CSA of vessel minus CSA of the device). If the conductivity of the saline solution is determined by calibration with various tubes of known CSA, then the calibration accounts for the dimension of the device and the calculated CSA corresponds to that of the total vessel lumen as desired. In at least one embodiment, the calibration of the CSA measurement system will be performed at 37° C. by applying 100 mmHg in a solid polyphenolenoxide block with holes of known CSA ranging from 7.065 mm² (3 mm in diameter) to 101.7 mm² (36 mm in diameter). If the conductivity of the solution(s) is/are obtained from a conductivity meter independent of the device, however, then the CSA of the device is generally added to the computed CSA to give the desired total CSA of the luminal organ.

The signals obtained herein are generally non-stationary, nonlinear and stochastic. To deal with: non-stationary stochastic functions, one may use a number of methods, such as the Spectrogram, the Wavelet's analysis, the Wigner-Ville distribution, the Evolutionary Spectrum, Modal analysis, or preferably the intrinsic model function (IMF) method. The mean or peak-to-peak values can be systematically determined by the aforementioned signal analysis and used to compute the Gₚ as referenced herein.

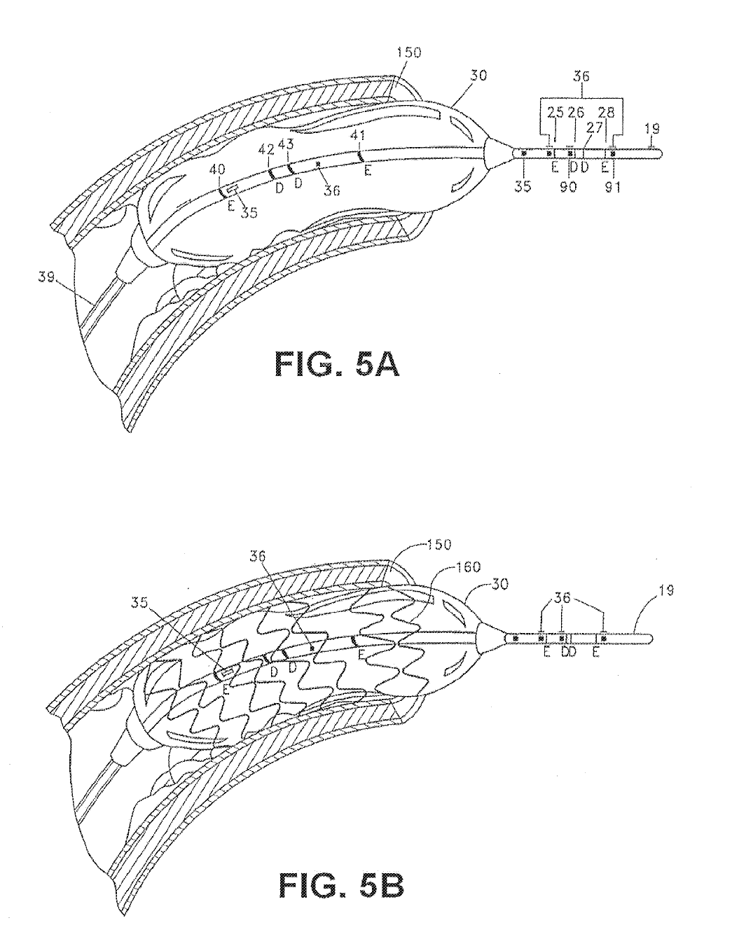

Referring to the embodiment shown in FIG. 5A, the angioplasty balloon 30 is selected. on the basis of Gₚ and is shown distended within a coronary artery 150 for the treatment of stenosis. As described above with reference to FIG. 2C, a set of excitation electrodes 40, 41 and detection electrodes 42, 43 are located within the angioplasty balloon 30. In another embodiment, and as shown in FIG. 5B, an angioplasty balloon 30 is used to distend a stent 160 within blood vessel 150.

In an additional exemplary approach, concomitant with measuring Gₚ and or pressure gradient at the treatment or measurement site, a mechanical stimulus is introduced by way of inflating a low or high pressure balloon based on high or low value of Gₚ, respectively. This action releases the stent from the device, thereby facilitating flow through the stenosed part of the organ. In another approach, concomitant with measuring Gₚ and or pressure gradient at the treatment site, one or more pharmaceutical substances for diagnosis or treatment of stenosis is injected into the treatment site. For example, an in at least one approach, the injected substance cars be smooth, muscle agonist or antagonist. In yet another approach, concomitant with measuring Gₚ and or pressure gradient at the treatment site, an inflating fluid is released into the treatment site for release of any stenosis or materials causing stenosis in the organ or treatment site.

For valve area determination, it is not generally feasible to displace the entire volume of the heart. Hence, the conductivity of blood is changed by injection, of a hypertonic saline solution into the pulmonary artery which will transiently change the conductivity of blood. If the measured total conductance is plotted versus blood conductivity on a graph, the extrapolated conductance at zero conductivity corresponds to the parallel conductance. In order to ensure that the two inner electrodes of the detector are positioned in the plane of the valve annulus (2-3 mm). In one exemplary embodiment, two pressure sensors are advantageously placed immediately proximal and distal to the detection electrodes (3-2 mm above and below, respectively) or several sets of detection electrodes (see, e.g., FIGS. 2E and 2G). The pressure readings will then indicate the position of the detection electrode relative to the desired site of measurement (aortic valve; aortic-ventricular pressure; mitral valve: left ventricular-atrial pressure; tricuspid valve: right atrial-ventricular pressure; pulmonary valve; right ventricular-pulmonary pressure). The parallel conductance at the site of annulus is generally expected to be small since the annulus consists primarily of collagen which has low electrical conductivity. In an additional application, a pull back or push forward through the heart chamber will show different conductance due to the change in geometry and parallel conductance. This can be established for normal patients which can then he used to diagnose valvular stenosis.

In an exemplary approach, for the esophagus or the urethra, the procedures can conveniently be done by swallowing fluids of known conductances into the esophagus and infusion of fluids of known conductances into the urinary bladder followed by voiding the volume. In another approach, fluids can be swallowed or urine voided followed by measurement of the fluid conductances from samples of the fluid. The latter method can be applied to the ureter where a catheter can be advanced up into the ureter and fluids can either be injected from a proximal port on the probe (will also be applicable in the intestines) or urine production can be increased and samples taken distal in the ureter during passage of the bolus or from the urinary bladder.

In another exemplary approach, concomitant with measuring the cross-sectional area and or pressure gradient at the treatment or measurement site, a mechanical stimulus is introduced by way of inflating the balloon or by releasing a stent from the catheter, thereby facilitating flow through the stenosed part of the organ. In another approach, concomitant with measuring the cross-sectional area and or pressure gradient at the treatment site, one or more pharmaceutical substances for diagnosis or treatment of stenosis is injected into the treatment site. For example, in one approach, the injected substance can he smooth muscle agonist or antagonist. In yet another approach, concomitant with measuring fee cross-sectional area and or pressure gradient at the treatment site, an inflating fluid is released into the treatment site for release of any stenosis or materials causing stenosis in the organ or treatment site.

Again, it is noted that the devices, systems, and methods described herein can be applied to any body lumen or treatment site. For example, the devices, systems, and methods described herein can be applied to any one of the following exemplary bodily hollow systems; the cardiovascular system including the heart, the digestive system, the respiratory system, the reproductive system, and the urogenital tract.

The various single injection methods 300 of the present disclosure offer a number of advantages over a two-injection method, including the reduction in the number of steps for the physician to perform (one injection instead, of two), and the overall reduction in time to perform a procedure. Furthermore, a single injection method 300 allows a physician to obtain the CSA at the same time as opposed to matching between the two injections, which involves fewer assumptions and is therefore more accurate. A single injection method 300 also allows for the reconstruction of the temporal variation of the CSA during the injection period, allowing for a mean, minimum or maximum CSA to be determined. In addition to the foregoing, a single injection method 300 reduces the signal processing to identify the point of injection since there is only one injection, and it is easier to identify and match the simultaneous signals since the two frequency-conductance curves occur on the same time domain. Furthermore, the techniques of the present disclosure are minimally invasive, accurate, reliable and easily reproducible.

While various embodiments of single injection systems useful to obtain parallel tissue conductance within luminal organs and methods for using the same have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure, indeed, this disclosure is not intended to be exhaustive or to limit the scope of the disclosure.

Further, in describing representative embodiments, the disclosure may have presented a method and/or process as a particular sequence of steps. However, to the extent that the method or process does not rely on the particular order of step set forth herein, the method or process should not be limited to the particular sequence of steps described. Other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A method of obtaining a cross-sectional area of a luminal organ using impedance, comprising the steps of:
positioning at least part of a detection device into a luminal organ at a first location, the detection device having a detector;
introducing a plurality of frequencies through the detector of the detection device within the luminal organ;
obtaining a plurality of conductance measurements using the detector of the device associated with the plurality of frequencies; and
determining a cross-sectional area of the luminal organ at the detector using a data acquisition and processing system in communication with the detection device and programmed to calculate the cross-sectional area, wherein the cross-sectional area is determined based in part upon the plurality of conductance measurements, a known distance between two detection electrodes of the detector of the detection device, and a conductivity of blood within the luminal organ.

2. The method of claim 1, further comprising the step of:
generating a size profile of the luminal organ using the calculated cross-sectional area.

3. The method of claim 1, wherein the conductivity of blood within the luminal organ is determined by operating the detector of the detection device within a catheter positioned within the luminal organ by obtaining a conductance measurement within the catheter having a known diameter.

4. The method of claim 1, wherein the step of positioning is performed by positioning the at least part of the detection device into the luminal organ wherein the detector comprises the two detection electrodes positioned in between two excitation electrodes, wherein the known distance between the two detection electrodes is at least 0.5 mm.

5. The method of claim 4, wherein the step of introducing the plurality of frequencies is performed by operating a frequency generator in communication with the detection device, and wherein the step of obtaining the plurality of conductance measurements is made by operating the two detection electrodes in an electric field generated by the two excitation electrodes while the plurality of frequencies are introduced within the luminal organ.

6. The method of claim 1, wherein the determining step is further performed to determine a parallel tissue conductance.

7. The method of claim 1, wherein the step of introducing a plurality of frequencies is performed by introducing a combined signal into the luminal organ, the combined signal comprising the plurality of frequencies.

8. The method of claim 7, wherein the step of obtaining the plurality of conductance measurements is performed by obtaining the plurality of conductance measurements associated with the combined signal comprising the plurality of frequencies.

9. The method of claim 8, wherein the step of determining the cross-sectional area of the luminal organ also comprises the step of deconvoluting the plurality of conductance measurements associated with the combined signal to determine separate conductance measurements for each frequency of the plurality of frequencies of the combined signal.

10. A method of obtaining a cross-sectional area of a luminal organ using impedance, comprising the steps of:
positioning at least part of a detection device having a detector into a luminal organ at a first location, the detector comprising two detection electrodes positioned in between two excitation electrodes, wherein a known distance between the two detection electrodes is at least 0.5 mm;
introducing a plurality of frequencies through the detector of the detection device within the luminal organ;
obtaining a plurality of conductance measurements using the detector of the device associated with the plurality of frequencies; and
determining a cross-sectional area of the luminal organ at the detector using a data acquisition and processing system in communication with the detection device and programmed to calculate the cross-sectional area, wherein the cross-sectional area is determined based in part upon the plurality of conductance measurements, the known distance between detection electrodes of the detector of the detection device, and a conductivity of blood within the luminal organ; and
generating a size profile of the luminal organ using the calculated cross-sectional area.
